(19) United States
(12) Patent Application Publication
(10) Pub. No.: US 2025/0017577 A1
(43) Pub. Date: Jan. 16, 2025

(54) TORQUE LIMITER AS WELL AS HANDLE UNIT AND SURGICAL INSTRUMENT WITH A TORQUE LIMITER

(71) Applicant: Ulrich WERNZ, Tuttlingen (DE)

(72) Inventor: Ulrich WERNZ, Tuttlingen (DE)

(21) Appl. No.: 18/771,362

(22) Filed: Jul. 12, 2024

(30) Foreign Application Priority Data
Jul. 13, 2023 (DE) 10 2023 118 540.3

(51) Int. Cl. A61B 17/00; A61B 90/00

(52) U.S. Cl. CPC A61B 17/00; A61B 2017/0046; A61B 2090/031

(57) ABSTRACT

A torque limiter, especially for a surgical instrument, with an axis of rotation includes an input element for the input of torque into the torque limiter, an output element for the output of torque from the torque limiter and a coupling unit having a coupling body by means of which the input element can be coupled with the output element. The coupling unit is arranged in the torque limiter so that it can be replaced. The preferred invention also includes handle unit with a torque limiter and a surgical instrument with a torque limiter.

### CROSS-REFERENCE TO RELATED APPLICATIONS

This application claims priority under 35 U.S.C. § 119 (b) to German Patent Application No. 10 2023 118 540.3, filed on Jul. 13, 2023, the disclosure of which is incorporated herein by reference in its entirety.

### BACKGROUND OF THE INVENTION

The preferred invention relates to a torque limiter as well as to a handle unit and a surgical instrument with such a torque limiter.

Torque limiters are known in many different forms. These torque limiters have torque limits that are fixed or adjustable. Typically, a torque limiter is configured such that a torque applied to the torque limiter on the input side is transferred only up to the torque limit to an output side of the torque limiter.

In medical applications, it is often important to maintain exact torque specifications, in order to guarantee the safety of implants and other medical products. Therefore, torque limiters are also used in surgical instruments. Such torque limiters are known, for example, from EP 2 311 397 B1 and EP 2 085 041 A1.

A disadvantage in known torque limiters is that the torque accuracy decreases with time such that the actual torque limit deviates from the desired torque limit in a no longer acceptable way. Surgical torque limiters, however, require high torque accuracy.

Therefore, for surgical instruments with torque limiters, a maximum lifetime is defined, for example, as a function of their age or their performed sterilization cycles, and after reaching this maximum lifetime, the instruments may no longer be used and must be recalibrated or disposed of.

This not only leads to a large amount of instrument waste, but also to high costs, especially because torque limiters for surgical instruments are usually very expensive systems.

Therefore, the preferred invention is based on the problem of disclosing a torque limiter, with which the service life of surgical instruments can be increased and which can be manufactured easily and economically.

The preferred invention is further based on the problem of disclosing a handle unit and a surgical instrument with the corresponding properties.

### BRIEF SUMMARY OF THE INVENTION

The problem is solved according to the preferred invention by a torque limiter with the features of the torque limiter claimed and described herein, including preferably having a handle unit with the features described and claimed herein, and a surgical instrument with the features described and claimed herein.

Advantageous constructions and refinements of the preferred invention are specified in the dependent claims.

A torque limiter according to the preferred invention, especially for a surgical instrument, comprises an axis of rotation, an input element for the input of torque into the torque limiter, an output element for the output of torque from the torque limiter, and a coupling unit having at least one coupling body, by means of which the input element can be coupled with the output element, wherein the coupling unit is arranged replaceable in the torque limiter. In this way, after reaching a defined lifetime, for example, a defined age or a defined number of sterilization cycles, the coupling unit can be replaced for a new, calibrated coupling unit. In this way, the assembly that is essential for defining an actual torque limit can be replaced without having to dispose of the entire torque limiter or the entire surgical instrument.

Preferably, the torque limiter is constructed such that a torque applied to the input element below the torque limit can be transferred to the output element. In contrast, an amount of the torque applied to the input element exceeding the torque limit is preferably not transferrable from the input element to the output element. The coupling body can be constructed for positive-locking and/or non-positive-locking torque transfer.

Preferably, the coupling unit can be replaced without a special assembly device and/or with a standard tool. The coupling unit can be replaced, in particular, without a tool. In an especially preferred way, the coupling unit is arranged in the torque limiter so that it is replaceable by the user. If the torque limiter is suitable and/or designed for a surgical instrument, medical professionals can be considered to be the average user group. Preferably, the coupling unit is constructed to be completely replaceable, that is, with all components belonging to the coupling unit.

The coupling unit can have a basic circular-ring-like shape arranged about the axis of rotation. Preferably, the at least one coupling body also has a basic circular-ring-like shape. Preferably, the input element is arranged with respect to the axis of rotation radially outside the coupling unit; the output element is arranged with respect to the axis of rotation radially inside the coupling unit.

The coupling unit can be arranged along the axis of rotation in and/or on the torque limiter. In this way, the structural design of the torque limiter can be simplified.

Preferably, the coupling unit has a first coupling element that can be coupled on one side with the at least one coupling body and on the other side with the input element or the output element in a rotationally locked and/or direct manner. Here, the first coupling element is preferably formed by a different component than the input element. In particular, if the first coupling element is arranged radially outside the at least one coupling body, the first coupling element can have a housing function for the coupling unit. In this way, the replaceability of the coupling unit can be improved. The rotationally locked coupling can be realized in the form of a positive-locking and/or non-positive-locking connection. A preferred embodiment of a rotationally locked coupling is here a positive-locking connection, in particular, by means of a spline.

The coupling unit can further have a second coupling element, which can be coupled on one side with the at least one coupling body as a function of the torque and/or directly and on the other side with the input element or the output element rotationally locked and/or directly. The torque limit of the torque limiter can be defined through the torque-dependent coupling by means of the active connection of the coupling body to the second coupling element. Here, the torque-dependent coupling can be realized by means of a preferably direct, positive-locking and/or non-positive-locking connection, which is overridden preferably by exceeding the torque limit according to the principle of a slipping clutch. Through the provision of the second coupling element, the resulting wear can occur, in particular, on the second coupling element, so that the service life of the input and output elements is not negatively affected. The rotationally locked coupling of the at least one coupling body with the input element or the output element is preferably realized by a positive-locking connection, for example, by means of a spline.

In one preferred embodiment of the invention, the at least one coupling body has at least one pressing element constructed preferably as a spring arm. Preferably, the at least one pressing element contacts the second coupling element and in this way produces the active connection between the at least one coupling body and the second coupling element. The spring arm can be separated from the rest of the coupling body by a gap that can be constructed, in particular, with an L-shaped form. The spring characteristics of the spring arm are preferably realized by means of the gap. The coupling body can have a plurality of pressing elements.

The second coupling element can have at least one latching element preferably constructed as a recess for interacting with the at least one pressing element. The contour of the at least one pressing element preferably corresponds at least in some sections to the contour of the at least one latching element. The second coupling element can have a plurality of latching elements. Preferably the second coupling element can have at least exactly as many latching elements as the at least one coupling body has pressing elements. The number of latching elements of the second coupling element can alternatively be a whole-number multiple of the number of pressing elements of the at least one coupling body.

According to one alternative embodiment of the invention, the at least one latching element is constructed as a roller body that is arranged in at least one recess of the second coupling element. Here, preferably one of the at least one roller bodies is arranged in each of the at least one recesses. The at least one roller body can be arranged so that it can rotate in the at least one recess. The at least one roller body preferably has a cylindrical shape. The at least one roller body and/or the at least one recess is preferably arranged parallel to the axis of rotation of the torque limiter. The second coupling element can have multiple roller bodies corresponding to the at least one roller body, which are preferably arranged uniformly distributed about the axis of rotation.

The at least one pressing element can be brought into direct active connection with the at least one latching element. In this way, the number of parts and the weight of the torque limiter can be reduced. Due to the lower number of parts, in particular, the replaceability of the coupling unit can be simplified or even made possible in the first place, for example, by preventing disengaged roller bodies. A continuous active connection between the at least one pressing element and the at least one latching element exists preferably in a latched state, that is, below the torque limit.

In one preferred embodiment of the invention, the at least one pressing element is formed by a plurality of pressing elements, which are aligned together in a same circumferential direction about the axis of rotation. In particular, if the at least one pressing element is constructed as a spring arm, in this way all spring arms are oriented in the same circumferential direction. For example, a first torque limit can be constructed with different magnitudes in a first circumferential direction about the axis of rotation relative to a second torque limit in a second circumferential direction about the axis of rotation. If the torque limiter is used, for example, in a screwdriver, then, for example, a maximum tightening torque can be defined to be less than a maximum loosening torque.

The at least one coupling body can be formed by multiple coupling bodies that are arranged along the axis of rotation one after the other. In this way, the production and assembly of the coupling unit can be simplified. The torque limit can be adjusted, in particular, at the factory, by the number of coupling bodies used. A retaining ring can be arranged on at least one axial end of the coupling unit for the axial securing of the at least one coupling body.

In one refinement of the preferred invention, the coupling unit has at least one spacer element that is preferably constructed as a rubber ring and that is arranged along the axis of rotation adjacent to one of the at least one coupling bodies. The at least one spacer element can be arranged at least in some sections in alternating arrangement with the at least one coupling body. The at least one spacer element can contribute to the damping, in particular, of the at least one pressing element, as well as to the reduction of noise. It can also cause an at least slight increase in the torque limit. In addition, the spacer element can form an axial assembly retaining element for the at least one torque body and can thus serve for simplifying assembly.

The coupling unit can be constructed such that it has an essentially mushroom-shaped outer contour. A top part of the mushroom-shaped outer contour can be especially good for gripping, which can make the replaceability of the coupling unit easier. In particular, if the coupling unit is constructed so that it can be replaced without a tool, it preferably has a mushroom-shaped contour. The mushroom-shaped outer contour is preferably formed such that one of the coupling elements, especially preferred the first coupling element, is constructed with a mushroom-shaped form.

In one embodiment of the preferred invention, the top part is arranged on the first coupling element or the second coupling element so that it can be removed in the axial direction. For this purpose, the top part can be arranged with a friction-locking and/or positive-locking connection on the first coupling element or the second coupling element. In particular, if the top part is constructed so that it can be removed, the retaining ring can be constructed in one piece with the first coupling element or the second coupling element. The retaining ring can have one or more through holes preferably arranged parallel to the axis of rotation.

The coupling unit is preferably constructed so that it can be latched and/or screwed in the input element and/or the output element. In particular, to construct the coupling unit so that it can be latched, the coupling unit can have on its outer contour an annular groove running preferably about the axis of rotation for engaging a latch projection. The latch projection can have a sealing element that is arranged on its side in a circumferential groove on the input element and/or the output element. The latch projection can thus have an additional sealing function.

A handle unit according to the preferred invention, in particular, for a surgical instrument, comprises a distal end, a proximal end, a handle longitudinal axis connecting the distal end and the proximal end, and a previously described torque limiter, wherein the input element has a gripping surface and the coupling unit is arranged in and/or on the proximal end. Here and in the following, the end of the handle unit that is arranged facing the user in the intended use of the handle unit is designated as the proximal end. Accordingly, here and in the following, the end of the handle unit that is arranged facing away from the user in the intended use of the handle unit is the distal end. In this way, in particular, unintentional removal of the coupling unit can be prevented and the introduction of dirt into the coupling unit and the whole handle unit can be reduced. The gripping surface preferably has an ergonomic shape, especially for a human hand, and can be constructed at least in some sections out of plastic, in particular, out of silicone. In particular, if the handle unit is designed and suitable for a surgical instrument, the output element preferably has a driveshaft for the surgical instrument.

In one preferred embodiment of the handle unit, the coupling unit can be arranged in the handle unit such that the axis of rotation is arranged along the handle longitudinal axis. In an especially preferred way, the axis of rotation is arranged on the handle longitudinal axis. A surgical instrument according to the preferred invention comprises an aforementioned torque limiter or an aforementioned handle unit.

### BRIEF DESCRIPTION OF THE SEVERAL VIEWS OF THE DRAWING

The foregoing summary, as well as the following detailed description of the preferred invention, will be better understood when read in conjunction with the appended drawings. For the purpose of illustrating the preferred invention, there are shown in the drawings embodiments which are presently preferred. It should be understood, however, that the invention is not limited to the precise arrangements and instrumentalities shown. In the drawings:

FIG. 1 is a side elevational view of a first embodiment of a handle unit with a torque limiter,

FIG. 2 is an exploded side perspective view of the embodiment of the handle shown in FIG. 1,

FIG. 3 is a magnified longitudinal cross-sectional view of the embodiment of the handle shown in FIG. 1, taken along a longitudinal axis of the handle of FIG. 1,

FIG. 4 is a magnified perspective cross-sectional view of a rear portion of the handle shown in FIG. 3,

FIG. 5 is a side perspective view of a coupling unit of the embodiment of the handle shown in FIG. 1,

FIG. 6 is an exploded side perspective view of the coupling unit shown in FIG. 5,

FIG. 7 is a side perspective cross-sectional view of the coupling unit of FIG. 5, taken along a longitudinal axis of the coupling unit shown in FIG. 5,

FIG. 8 is a front perspective, cross-sectional view of the coupling unit shown in FIG. 5,

FIG. 9 is a side perspective, exploded view of a second embodiment of a handle unit with a torque limiter,

FIG. 10 is a side perspective, cross-sectional view of the embodiment of the handle shown in FIG. 9,

FIG. 11 is a side perspective, exploded view of an alternative embodiment of a coupling unit,

FIG. 12 is a side perspective, cross-sectional view of the coupling unit shown in FIG. 11, taken along a longitudinal axis of the coupling unit of FIG. 11, and

FIG. 13 is a front elevational, cross-sectional view of the coupling unit shown in FIG. 11, taken generally perpendicular to the longitudinal axis of the coupling unit of FIG. 11.

### DETAILED DESCRIPTION OF THE INVENTION

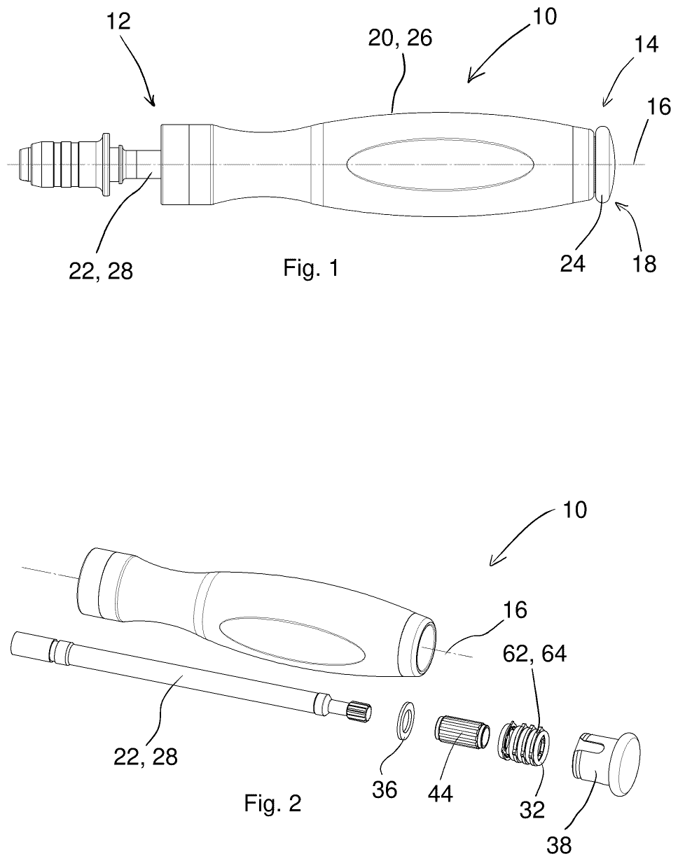

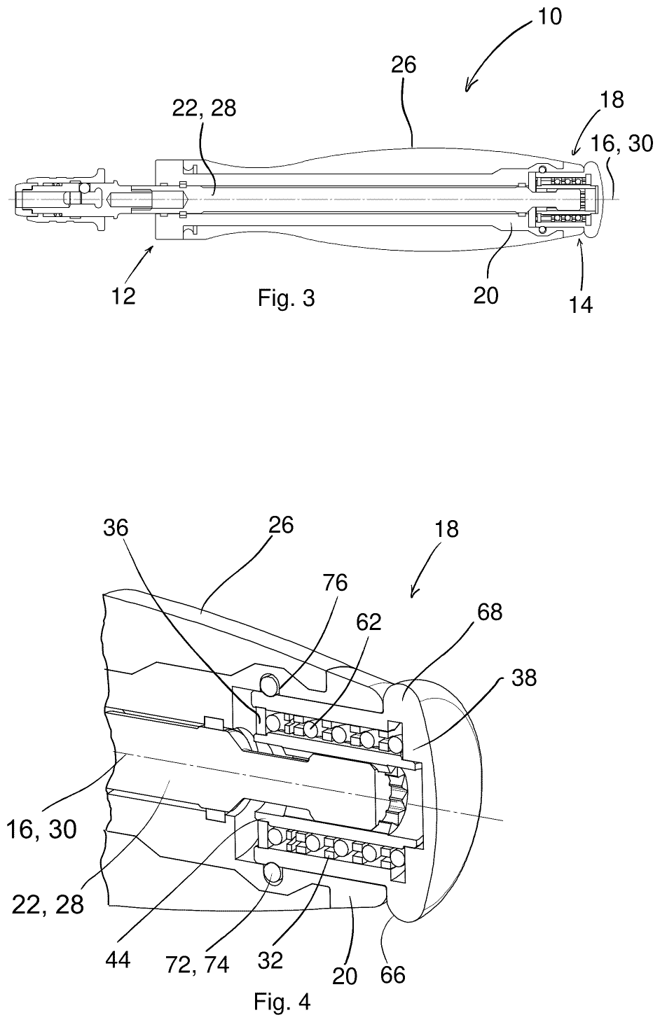

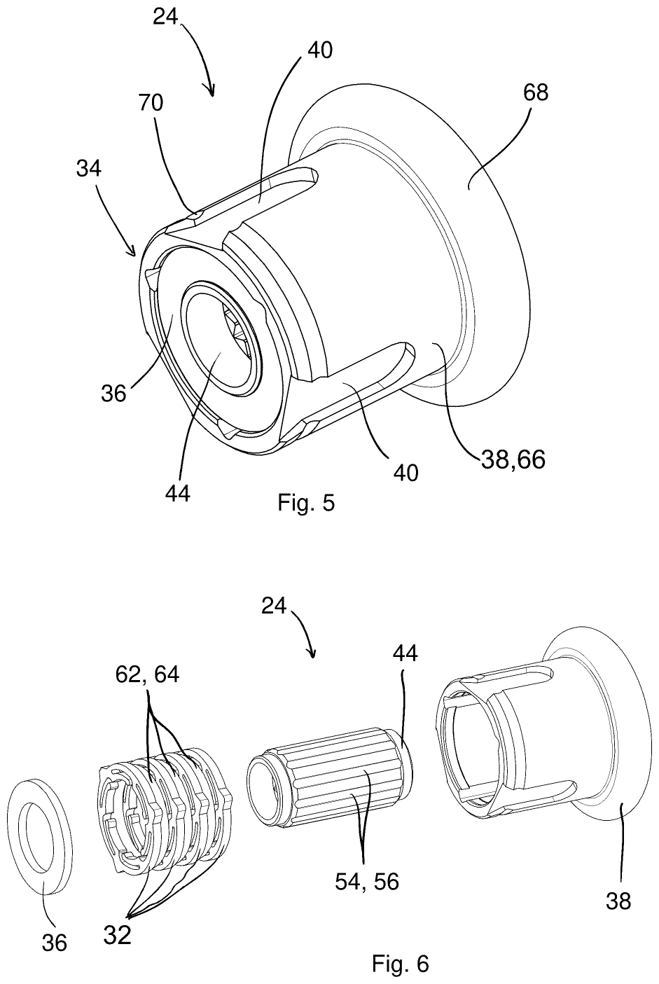

FIGS. 1 to 13 show different views of different preferred embodiments of the present invention. For the sake of clarity, not all reference symbols are used in each figure. The same reference symbols are used for the same parts.

In FIGS. 1-4, a first embodiment of a handle unit 10 according to the preferred invention is shown, which is designed and suitable, in particular, for a surgical instrument. The handle unit 10 has a distal end 12, a proximal end 14, and a grip longitudinal axis 16 connecting the distal end 12 and the proximal end 14. The handle unit 10 further comprises a torque limiter 18 with an input element 20 for the input of torque into the torque limiter 18, an output element 22 for the output of torque out of the torque limiter 18, and a coupling unit 24, by means of which the input element 20 can be coupled with the output element 22. The coupling unit 24 and its arrangement in the handle unit 10 are shown in more detail, in particular, in FIGS. 4-8.

In the illustrations in FIGS. 1-4, it can be seen especially well that the input element 20 has a gripping surface 26, and the coupling unit 24 is arranged in and on the proximal end 14 of the handle unit 10. The gripping surface 26 preferably has an ergonomic shape and can be constructed at least in some sections out of plastic, in particular, out of silicone. In particular, if the handle unit 10 is designed and suitable for a surgical instrument, the output element 22 preferably has a driveshaft 28 for the surgical instrument. In one preferred embodiment of the handle unit 10, the coupling unit 24 can be arranged in the handle unit 10 such that an axis of rotation 30 of the torque limiter 18 is arranged along the handle longitudinal axis 16. In an especially preferred way, the axis of rotation 30 is arranged on the handle longitudinal axis 16.

The coupling unit 24 is preferably arranged replaceable in the torque limiter 18. Preferably, the coupling unit 24 can be replaced without an assembly device and/or with a standard tool. The coupling unit 24 can be replaceable, in particular, without a tool. In an especially preferred way, the coupling unit 24 is arranged in the torque limiter 18 so that it can be replaced by the user. Preferably, the coupling unit 24 is constructed so that it is completely replaceable, that is, with all of its components belonging to the coupling unit 24.

Preferably the torque limiter 18 is constructed such that the torque applied on the input element 20 below the torque limit can be transferred from the input element 20 to the output element 22. In contrast, an amount of the torque applied to the input element 20 exceeding the torque limit preferably cannot be transferred from the input element 20 to the output element 22.

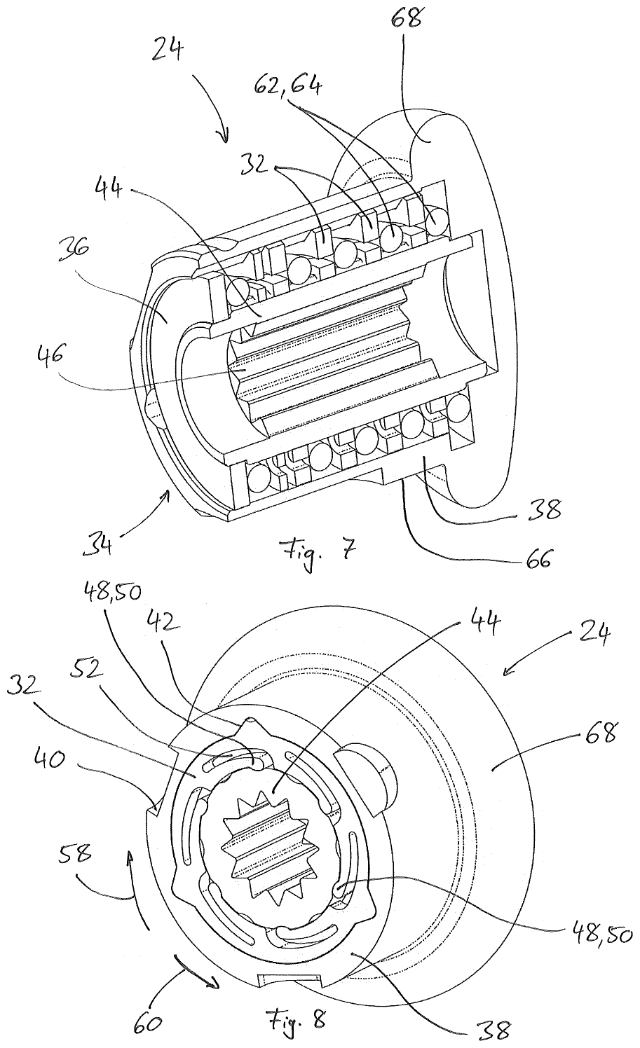

As FIGS. 2 and 6, among others, show, the coupling unit 24 has at least one coupling body 32, which can be constructed for positive-locking and non-positive-locking torque transfer. The at least one coupling body 32 is preferably formed by multiple coupling bodies 32, in the present embodiment by four coupling bodies 32. The coupling bodies 32 can be arranged along the axis of rotation 30 one after the other. The torque limit can be adjusted, in particular, at the factory, by the number of coupling bodies 32 used. A retaining ring 36 for the axial securing of the coupling body 32 can be arranged on at least one axial end 34 of the coupling unit 24.

The coupling unit 24 can have a basic circular-ring shape arranged about the axis of rotation 30. Preferably, in this way, each of the coupling bodies 32 also has a basic circular-ring shape. Preferably, the input element 20 is here arranged with respect to the axis of rotation 30 radially outside of the coupling unit 24, and the output element 22 is arranged with respect to the axis of rotation 30 radially inside the coupling unit 24 (FIG. 4). In particular, to simplify the structural design of the torque limiter 18, the coupling unit 24 can be arranged along the axis of rotation 30 in the torque limiter 18.

The coupling unit 24 is shown in more detail in FIGS. 4-8. The coupling unit 24 preferably has a first coupling element 38, which can be coupled rotationally locked and direct, as FIG. 8 shows, on one side with the coupling body 32 and on the other side by means of longitudinal grooves 40 with the input element 20. FIG. 4 here shows that the first coupling element 38 is preferably formed by a different component from the input element 20 and is coupled directly with the input element 20. A preferred embodiment of the rotationally locked coupling is a positive-locking connection by means of a first spline 42.

In particular, if, as is preferably the case in the shown embodiment, the first coupling element 38 is arranged radially outside the coupling body 32, the first coupling element 38 can have a housing function for the coupling unit 24. In this way, the replaceability of the coupling unit 24 can be improved.

The coupling unit 24 can further have a second coupling element 44 that is also shown, among others, in FIG. 8. The second coupling element 44 can be coupled dependent on torque and directly on one side with the coupling bodies 32 and rotationally locked and direct on the other side with the output element 22. The rotationally locked coupling of the second coupling element 44 with the output element 22 is preferably also realized by a positive-locking connection, namely by means of a second spline 46 (see in particular FIG. 4).

The torque limit of the torque limiter 18 can be defined by means of the active connection of the coupling body 32 with the second coupling element 44 through the torque-dependent coupling. The torque-dependent coupling can here be realized by means of a direct positive-locking and non-positive-locking connection, which is preferably overridden by exceeding the torque limit according to the principle of a slipping clutch. Through the provision of the second coupling element 44, in particular, the resulting wear can occur on the second coupling element 44, so that the service life of the output element 22 is therefore not negatively affected.

For realizing the torque-dependent coupling of the coupling body 32 with the second coupling element 44, preferably each of the coupling bodies 32 has at least one pressing element 50 constructed as a spring arm 48. In an especially preferred way, each of the coupling bodies 32 has a plurality of spring arms 48, for example, six spring arms 48, which can be seen well, for example, in FIG. 8. Preferably each of the spring arms 48 contacts the second coupling element 44 and in this way creates the active connection between each coupling body 32 and the second coupling element 44. Each of the spring arms 48 can be separated from the rest of the coupling body 32 by a gap 52, which can be constructed, in particular, with an L-shaped form. The spring characteristics of each spring arm 48 is preferably realized by means of the gap 52.

The second coupling element 44 can have at least one latching element 56 constructed preferably as a recess 54 for interacting with one of the spring arms 48. The contour of each of the spring arms 48 preferably corresponds at least in some sections to the contour of the at least one recess 54. The second coupling element 44 can have a plurality of recesses 54. The number of recesses 54 of the second coupling element 44 can be a whole-number multiple, for example, twice, the number of spring arms 48 of one of the coupling bodies 32. Thus, each of the spring arms 48 can be brought into direct active connection with one of the recesses 54. A continuous active connection between the spring arms 48 and the recesses 54 preferably exists in a latched state, that is, below the torque limit.

As is shown, in particular, in FIG. 8, the spring arms 48 can all be oriented in a first circumferential direction 58 about the axis of rotation 30. Thus, a first torque limit can have a different magnitude in the first circumferential direction 58 about the axis of rotation 30 compared with a second torque limit in a second circumferential direction 60 about the axis of rotation 30. If the torque limiter 18 is used, for example, in a screwdriver, for example, a maximum tightening torque can be set lower than a maximum loosening torque. In the shown embodiment, the torque limit is higher for a rotation of the coupling body 32 relative to the second coupling element 44 in the first circumferential direction 58 than in the second circumferential direction 60.

As, in particular, FIGS. 4 and 7 show, the coupling unit 24 has preferably multiple spacer elements 64 that are constructed as rubber rings 62 and are arranged along the axis of rotation 30 in alternating fashion with the coupling bodies 32. The rubber rings 62 can contribute to the damping, in particular, of the spring arms 48, as well as to the reduction of noise, as well as to an, at least slight, increase in the torque limit. In addition, each of the rubber rings 62 can be an axial assembly retainer for the previously mounted coupling body 32.

FIGS. 4-8 primarily show that the coupling unit 24 can be constructed such that it has an essentially mushroom-shaped outer contour 66. In the assembled state of the coupling unit 24, the mushroom shape is preferably arranged about the axis of rotation 30 of the torque limiter 18. A top part 68 of the mushroom-shaped outer contour 66 can be particularly easy to grip, which can simplify the replaceability of the coupling unit 24. The mushroom-shaped outer contour 66 is preferably formed such that the first coupling element 38 is constructed with a mushroom-shaped form.

The coupling unit 24 can be constructed so that it can be latched and/or screwed into the input element 20 and/or the output element 22. In particular, to construct the coupling unit 24 so that it can be latched, the coupling unit 24 can have on its outer contour an annular groove 70 running preferably about the axis of rotation 30 for engaging a latch projection 72. The latch projection 72 can comprise a sealing element 74, which is arranged on its side in a circumferential groove 76 on the input element 20. Thus, the latch projection 72 can have an additional sealing function.

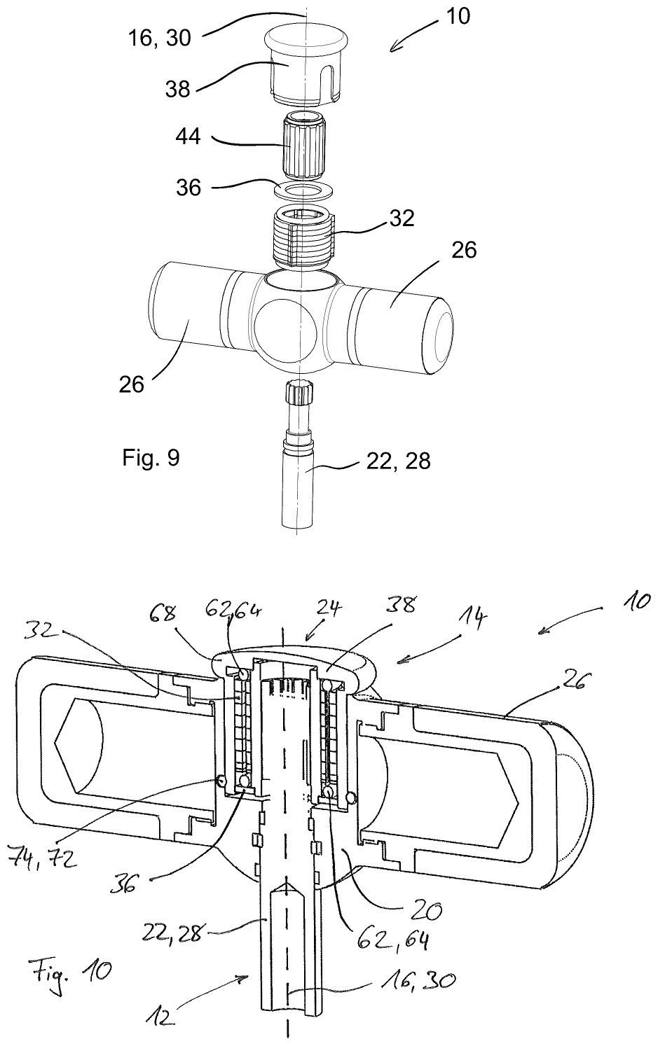

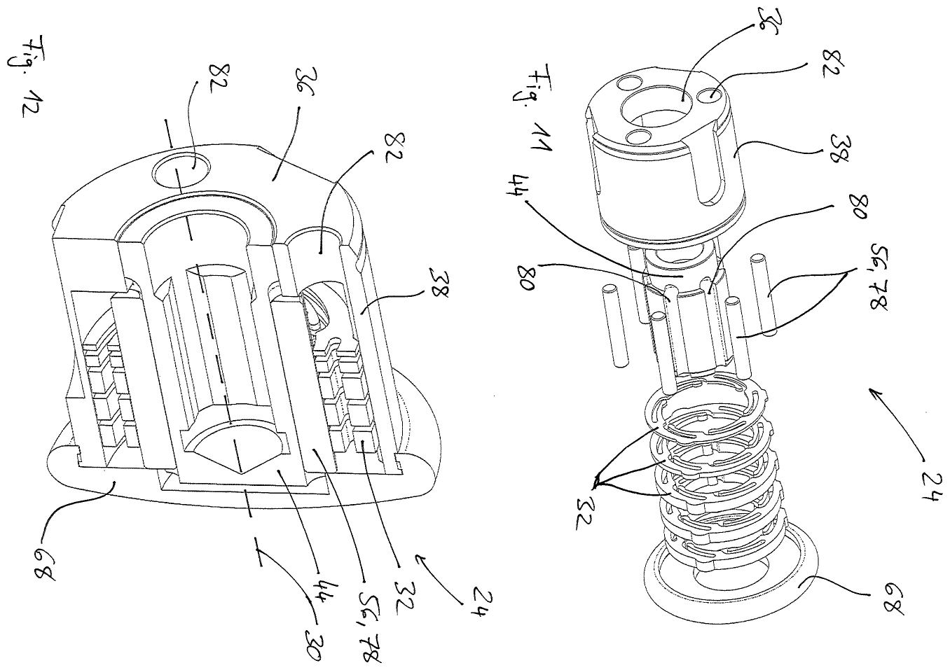

FIGS. 9-10 show a second embodiment of a handle unit, which is constructed largely corresponding to the embodiment shown in FIGS. 1-8. Therefore, below only the differences in comparison with the first embodiment will be discussed.

As can be seen from FIGS. 9 and 10, the handle unit 10 can have a T-shaped form, in particular, in that the gripping surface 26 is arranged perpendicular to the handle longitudinal axis 16. The shape of the input element 20 can be adapted accordingly. Such embodiments of the handle unit 10 can be used, for example, for applications in which a high torque is to be introduced into the handle unit 10.

In particular, FIG. 10 shows that the coupling body 32 arranged in the torque limiter 18 can be arranged directly adjacent to each other. The spacer elements 62, 64 can be arranged such that they seal the package formed from the coupling bodies 32 along the handle longitudinal axis 16 in both axial directions. Despite this distinguishing feature in comparison to the first embodiment, the basic structural design of the coupling unit 24 and/or the interfaces, in particular, the first coupling element 38 and the second coupling element 44, preferably do not differ between the first embodiment and the second embodiment. In this way, additional possibilities of the replaceable coupling unit 24 become clear: the same coupling unit 24 can be arranged in different handle units 10 and the same handle unit 10 can be equipped with different coupling units 24.

FIGS. 11-13 show an alternative embodiment of the coupling unit 24. The coupling unit 24 of FIGS. 11-13 differs from the coupling unit 24 shown in FIGS. 1-10 in that, among other things, the latching elements 56 are preferably constructed as cylindrical roller bodies 78. The roller bodies 78 are preferably parallel to the axis of rotation 30 of the torque limiter 18 and distributed uniformly about the axis of rotation 30. Preferably, the second coupling element has a number of recesses 80 corresponding to the number of roller bodies 78, wherein one of the roller bodies 78 can be arranged in each of the recesses 80.

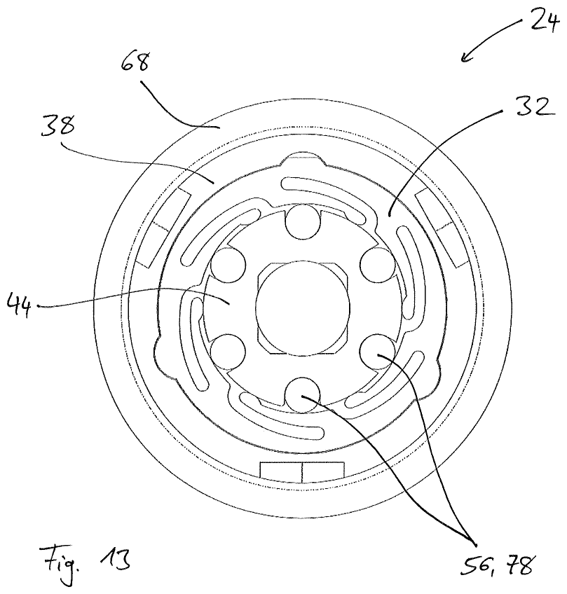

As, in particular, FIG. 12 shows, the top part 68 can be arranged axially removable on the first coupling element 38. For this purpose, the top part 68 can be arranged with a friction-locking and/or positive-locking connection on the first coupling element 38. Furthermore, the retaining ring 36 can be constructed in one piece with the first coupling element 38. Thus, the first coupling element 38 can be constructed with a pot-shaped form. The retaining ring 38 can have one or more through holes 82 preferably arranged parallel to the axis of rotation 30.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the preferred present invention as defined by the appended claims.

### LIST OF REFERENCE SYMBOLS

10 Handle unit
12 Distal end
14 Proximal end
16 Handle longitudinal axis
18 Torque limiter
20 Input element
22 Output element
24 Coupling unit
26 Gripping surface
28 Driveshaft
30 Axis of rotation
32 Coupling body
34 Axial end
36 Retaining ring
38 First coupling element
40 Longitudinal groove
42 First spline
44 Second coupling element
46 Second spline
48 Spring arm
50 Pressing element
52 Gap
54 Recess
56 Latching element
58 First circumferential direction
60 Second circumferential direction
62 Rubber ring
64 Spacer element
66 Outer contour
68 Top part
70 Annular groove
72 Latch projection
74 Scaling element
76 Circumferential groove
78 Roller body
80 Recess
82 Through hole

1. A torque limiter, especially for a surgical instrument with an axis of rotation, the torque limiter comprising:
an input element to input torque into the torque limiter,
an output element for the output of torque from the torque limiter, and
a coupling unit having a coupling body, by means of which the input element can be coupled with the output element, the coupling unit is arranged in the torque limiter so that the output element can be replaced.

2. The torque limiter according to claim 1, characterized in that the coupling unit is arranged along the axis of rotation in and on the torque limiter.

3. The torque limiter according to claim 1, characterized in that the coupling unit has a first coupling element which can be one of coupled, rotationally locked and directly connected on one side with the coupling body and on an opposite side with the input element.

4. The torque limiter according to claim 3, characterized in that the coupling unit has a second coupling element, which can be coupled on one side with the coupling body and on an opposite side with the input element.

5. The torque limiter according to claim 1, characterized in that the coupling body has a pressing element constructed as a spring arm.

6. The torque limiter according to claim 4, characterized in that the second coupling element has a latching element for interacting with a pressing element of the coupling body.

7. The torque limiter according to claim 6, characterized in that the latching element is constructed as a roller body that is arranged in a recess of the second coupling element.

8. The torque limiter according to one of claim 5 to 7, characterized in that the pressing element is formed by a plurality of pressing elements that are all oriented in a same circumferential direction about the axis of rotation.

9. The torque limiter according to claim 1, characterized in that the coupling body is formed by multiple coupling bodies that are arranged one after the other along the axis of rotation.

10. The torque limiter according to claim 9, characterized in that the coupling unit comprises a spacer element that is constructed as a rubber ring and is arranged along the axis of rotation adjacent to one of the multiple coupling bodies.

11. The torque limiter according to claim 1, characterized in that the coupling unit has an essentially mushroom-shaped outer contour.

12. The torque limiter according to claim 1, characterized in that the coupling unit is configured to be one of latched and screwed into the input element.

13. A handle unit, especially for a surgical instrument, the handle unit comprising:
a distal end,
a proximal end,
a handle longitudinal axis connecting the distal end and the proximal end, and
a torque limiter having an input element to input torque into the torque limiter, an output element and a coupling unit, wherein the input element has a gripping surface and the coupling unit is arranged one of in and on the proximal end.

14. The handle unit according to claim 13, characterized in that the coupling unit is arranged in the handle unit such that an axis of rotation of the torque limiter is arranged along the handle longitudinal axis.

15. (canceled)

16. The torque limiter of claim 1, characterized in that the coupling unit has a first coupling element which can be one of coupled, rotationally locked and directly connected on one side with the coupling body and on an opposite side with the output element.

17. The torque limiter of claim 3, characterized in that the coupling unit has a second coupling element which can be coupled on the opposite side with the output element.

18. The torque limiter of claim 1, characterized in that the coupling unit is configured to be one of latched and screwed into the output element.
